(19) United States
(12) Patent Application Publication
(10) Pub. No.: US 2005/0192355 A1
(43) Pub. Date: Sep. 1, 2005

(54) Compositions of non-steroidal anti-inflammatory drugs and decongestants or anti-histamines

(75) Inventor: Roger G. Berlin, Mendham, NJ (US)

Correspondence Address: DARBY & DARBY P.C., P.O. BOX 5257, NEW YORK, NY, 10150-5257, US

(73) Assignee: Wyeth, Madison, NJ (US)

(21) Appl. No.: 11/055,338

(22) Filed: Feb. 10, 2005

Related U.S. Application Data
(60) Provisional application No. 60/545,458, filed on Feb. 17, 2004.

(51) Int. Cl. A61K 31/192; A61K 31/137

(52) U.S. Cl. 514/570; 514/649

(57) ABSTRACT

The present invention relates to improved dosage forms of pharmaceuticals for treating rhinitis associated with allergies and colds. The dosage forms include an effective amount each of a non-steroidal anti-inflammatory drug, and a decongestant or an antihistamine wherein the effective amount of the decongestant or antihistamine is less than 75% of an amount present in an approved dose of the decongestant or the antihistamine relative to an amount of the NSAID corresponding to about 100% of the amount present in a normal strength dosage form of the NSAID.

### FIELD OF THE INVENTION

The present invention relates to improved dosage forms of pharmaceuticals for treating rhinitis associated with allergies and colds.

### BACKGROUND OF THE INVENTION

Decongestants and Antihistamines Rhinitis refers to an inflammatory disorder of the nasal passages. The symptoms of rhinitis typically consist of sneezing, rhinorrhea, nasal congestion, and increased nasal secretions. Untreated rhinitis may lead to other disorders including infection of the sinuses, ears and lower respiratory tract.

Two types of oral medication are commonly used to treat rhinitis: decongestants and antihistamines. Decongestants and antihistamines differ in mechanism of action, therapeutic effects, and side effects.

Decongestants agents act to constrict vessels in the nasal mucus membranes and thereby decrease tissue swelling and nasal congestion. Decongestants are found to be better than antihistamines for relieving such nasal congestion and restoring free flow of air in the nasal passages. However decongestants are stimulatory and may produce nervousness, restlessness, and insomnia, especially if taken at night.

Antihistamines act to block the binding of histamine to histamine receptors in the nasal membranes. Histamine is a mediator released from cells which line the walls of the nasal mucous membranes (mast cells). When released, histamine is known to bind to local receptors and thereby cause sneezing, nasal itching, swelling of the nasal membranes, and increased nasal secretions. Accordingly, by blocking the binding of histamine, antihistamines relieve these effects. However, antihistamines are sedating and often impair mental acuity and cause involuntary sedation.

Non-steroidal anti-inflammatory drugs (NSAIDS) have an analgesic, anti-inflammatory, and antipyretic activity and as such are ideally suited for use in cold formulations. NSAIDS have a low incidence of untoward side effects.

Exemplary cold formulations containing NSAIDS and a decongestant include Advil Cold and Sinus™, Motrin Cold and Flu™, Motrin IB Sinus™ and Dristan Sinus™, each containing 200 mg ibuprofen and 30 mg pseudoephedrine.

U.S. Pat. No. 5,025,019 teaches pharmaceutical compositions and methods of using a composition containing a NSAID in combination with at least one other active component selected from an antihistamine, decongestant, cough suppressant or expectorant.

While these combination products provide effective symptom treatment of rhinitis due to cold and allergy, they do not alleviate the unfavorable side effects of decongestants or antihistamines in sensitive individuals. Thus there remains a need in the art to treat symptoms of rhinitis but reduce the side effects such as restlessness, nervousness, and insomnia, from treatment with decongestant or impairment of mental acuity and involuntary sedation, from treatment with an antihistamine.

### SUMMARY OF THE INVENTION

The present invention is directed to a pharmaceutical composition which includes an effective amount of each of a non-steroidal anti-inflammatory drug (NSAID), a decongestant, or an antihistamine, wherein the effective amount of the decongestant or the antihistamine is less than about 75% of an amount present in an approved dose of the decongestant or the antihistamine, relative to an amount of the NSAID corresponding to about 100% of the amount present in a normal strength dosage form of the NSAID.

An embodiment of the invention is a pharmaceutical composition containing from about 10 to about 60 mg pseudoephedrine hydrochloride per dosage unit, from about 1 to about 4 mg chlorpheniramine per dosage unit, and from about 200 to about 400 mg ibuprofen per dosage unit.

The present invention is further directed to a method of relieving symptoms of rhinitis in a mammal. This method comprises administering an antihistaminic effective amount of an antihistamine or a decongestive effective amount of a decongestant, and an anti-inflammatory effective amount of a non-steroidal anti-inflammatory agent, wherein the effective amount of the decongestant or the antihistamine is less than about 75% of an amount present in an approved dose of the decongestant or the antihistamine, relative to an amount of the NSAID corresponding to about 100% of the amount present in a normal strength dosage form of the NSAID. Preferably in accordance with this method the antihistamine, the decongestant and the non-steroidal anti-inflammatory agent are present in a single dosage form. However, they can also be provided in separate dosage forms for administration together, or as separate dosage forms with instructions of how to achieve the effective dosages of the invention.

In addition to the two components, an anti-tussive agent can also be delivered in accordance with this invention.

### DETAILED DESCRIPTION OF THE INVENTION

It has been discovered that the addition of a non-steroidal anti-inflammatory agent to a composition containing an antihistamine or a decongestant enhances the efficacy of the antihistamine or decongestant, thus permitting a reduction in the total dose of antihistamine or decongestant. In particular, the amount of antihistamine or the decongestant can be less than or equal to about 75% of an amount present in an approved dose of the decongestant or the antihistamine relative to an amount of the NSAID corresponding to about 100% of the amount present in a normal strength dosage form of the NSAID. This combination of a 100% dose of an NSAID with a reduced dose of an antihistamine or a decongestant or both results in an enhanced effect of the decongestant or antihistamine. This novel combination of a non-steroidal anti-inflammatory agent with reduced levels of antihistamine or decongestant provides the same or greater symptomatic relief of rhinitis, including allergy, cold, cold-like, and flu symptoms, as conventional products containing the higher amounts of antihistamine and decongestants. This discovery is particularly advantageous because lowering the amounts of antihistamine or decongestant lowers the unwanted side effects of each of these ingredients, such as the stimulatory effect of decongestants, and the sedating effect of antihistamines.

As used herein, the term “rhinitis” refers to inflammation of the nasal mucous membranes, which could result from a cold, flu, or allergies. Rhinitis may be characterized by one or more cold-like symptoms.

Cold-like symptoms as used herein refers to coryzea, nasal congestion, upper respiratory infections, allergic rhinitis, otitis, sinusitis, and the like. Runny nose and nasal congestion can also be cold symptoms.

The terms “effective amount” or “therapeutically effective amount” of an active agent as provided herein is defined as an amount of the agent at least sufficient to provide the desired therapeutic effect. As noted above, the present invention is based on the discovery that the effective dose of a decongestant or antihistamine can be reduced if administered with a normal dose of a NSAID. The exact amount required will vary from subject to subject, depending on age, general condition of the subject, the severity of the condition being treated, and the particular active agent administered, and the like.

The term “normal approved dose” of an active agent as provided herein is defined as an amount of the agent that has been approved as safe and effective by the United States Food and Drug Administration for administration in humans in a particular dosage form. An approved dose is thus a dose found in a pharmaceutical product, an amount of active agent per unit dosage form. In the present invention, reference to a ratio of approved doses means doses approved for the same patient population (e.g., adult to adult or pediatric to pediatric), and approved for the same dosage form (e.g., elixir, tablet, capsule, caplet, controlled release, etc.).

In the practice of the invention, one of ordinary skill in the art can take an approved dosage form of any over-the-counter (OTC) or prescription decongestant or antihistamine, reduce it by, e.g., 25% to 50% or more, and co-administer it with an approved amount (dose) of a NSAID to achieve effective relief of rhinitis with reduced side effects. In one embodiment, the present invention contemplates the use of less than or equal to about 75% and more than 1% of an amount present in an approved dose of the decongestant or the antihistamine, relative to an amount of the NSAID corresponding to about 100% of the amount present in a normal strength dosage form of the NSAID. The present invention further contemplates the use of 10% to 65%, 30% to 55%, and 35% to 50%, of an amount present in an approved does of the decongestant or the antihistamine, relative to an amount of the NSAID corresponding to 100% of the amount present in a normal strength dosage form of NSAID.

While the present invention contemplates compositions comprising two components (i.e., decongestant and NSAID without antihistamine, or antihistamine and NSAID without decongestant), with the decongestant or antihistamine, present in a lower amount relative to conventional OTC decongestant or antihistamine products on the market, one can readily achieve the present invention by reducing the dose of the conventional antihistamine or decongestant products on the market when they are administered with a product containing a normal dose of a NSAID. Such reductions can be achieved by cutting an adult dose in half, e.g., administering half the amount of elixir or cutting a tablet in half, or by using a reduced dosage form, such as a decongestant or antihistamine product formulated for children in combination with an adult formulation of an NSAID at the approved dose.

### Antihistamines

The term “antihistamine,” used in connection with treating nasal symptoms associated with allergy or cold, generally refers to histamine H₁ receptor antagonists. Numerous chemical substances are known to have histamine H₁ receptor antagonist activity. Many useful compounds can be classified as ethanolamines, ethylenediamines, alkylamines, phenothiazines or piperidines. Representative H₁ receptor antagonists, include, without limitation: astemizole, azatadine, azelastine, acrivastine, brompheniramine, chlorpheniramine, clemastine, cyclizine, carebastine, cyproheptadine, carbinoxamine, descarboethoxyloratadine (also known as SCH-34117), desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum.

Of the foregoing histamine H₁ receptor antagonists, chlorpheniramine is specifically exemplified herein. The FDA approved adult dosage of chlorpheniramine is 4 mg orally every 4 to 6 hours as needed, up to a maximum of 24 mg per day. The FDA approved pediatric dosage of chlorpheniramine is 2 mg orally every 4 to 6 hours, up to a maximum of 12 mg per day. The preferred salt is chlorpheniramine maleate. In accordance with the present invention, the preferred adult dosage is thus reduced to 3 mg, 2 mg or 1 mg, orally every 4 to 6 hours as needed, up to a maximum of 6 to 18 mg per day. Similarly, in an embodiment of the invention, the pediatric dosage is 1.5 mg, 1 mg, or 0.5 mg, orally every 4 to 6 hours, up to a maximum of 3 to 9 mg per day. In a further embodiment, the invention permits combining a pediatric dosage of chlorpheniramine with an adult dosage of an NSAID, such as ibuprofen.

### Decongestants

The decongestants for use in the pharmaceutical compositions and methods of use of the present invention include, but are not limited to, pseudoephedrine, phenylephedrine, phenylpropanolamine. One of skill in the art would know of many other appropriate decongestants and their approved dosages.

The exemplified decongestant is pseudoephedrine. The usual adult dose of pseudoephedrine is 60 mg every 4 to 6 hours, up to a maximum of 240 mg per day. The usual pediatric dose of pseudoephedrine is 15 mg every 6 hours, up to a maximum of 60 mg per day for ages 2 to 5 and 30 mg every 6 hours, up to a maximum of 120 mg per day for ages 6 to 12. Thus, in specific embodiments of the practice of the present invention, the adult dose can be reduced to 45, 30, or 15 mg every 4 to 6 hours, with a maximum of 90 to 180 mg per day, and the pediatric dose can be reduced to about 11, 7.5 or 3.7 mg every 6 hours, up to a maximum of 11 to 45 mg per day.

### NSAIDs

The non-steroidal anti-inflammatory drugs (NSAID's) for use in the pharmaceutical compositions and methods of use of the present invention may be selected from any of the following categories:
(1) The propionic acid derivatives;
(2) the acetic acid derivatives;
(3) The fenamic acid derivatives
(4) The biphenylcarboxylic acid derivatives;
(5) The oxicams, and
(6) Cox-2 inhibitors

Accordingly, the term “NSAID” as used herein is intended to mean any non-steroidal anti-inflammatory compound, including the pharmaceutically acceptable non-toxic salts thereof, falling within one of the six structural categories above.

The specific compounds falling within the foregoing definition of the non-steroidal anti-inflammatory drugs for use in the present invention are well known to those skilled in the art and reference may be found in various literature reference sources for their chemical structures, pharmacological activities, side effects, normal dosage ranges, etc. See, for example, Physician's Desk Reference, and The Merek Index.

Of the propionic acid derivatives for use herein, ibuprofen, naxproxen, flurbiprofen, fenoprofen, ketoprofen, suprofen, fenbufen, and fluprofen may be mentioned as exemplified compounds. Of the acetic acid derivatives, exemplary compounds include tolmetin sodium, zomepirac, sulindac and indomethacin. Of the fenamic acid derivatives, exemplary compounds include mefenamic acid and meclofenamate sodium. Exemplary biphenylcarboxlic acid derivatives for use in the present invention include diflunisal and flufenisal. Exemplary oxicams include piroxicam, sudoxicam and isoxicam. Exemplary Cox-2 inhibitors include celecoxib, rofecoxib, meloxicam, and nimesulide. Of the foregoing non-steroidal anti-inflammatory drugs, in the practice of the exemplified embodiments of the present invention, ibuprofen is exemplified.

With respect to the dosage amount of the non-steroidal anti-inflammatory drugs in the compositions of the invention, although the specific dose will vary depending upon the age and weight of the patient, the severity of the symptoms, the incidence of side effects and the like, for humans, typical effective analgesic amounts of NSAID's are about 100-500 mg diflunisal, about 25-100 mg zomepirac sodium, about 50-450 mg ibuprofen, more preferably 100-250 mg ibuprofen, about 125-500 mg naproxen, about 25-100 mg flurbiprofen, about 50-199 mg fenoprogen, about 10-20 mg piroxicam, about 125-250 mg mefanaic acid, about 100-400 mg fenbufen or about 25-50 mg ketoprofen; however, greater or lesser amounts may be employed if desired or necessary.

### Anti-Tussitives

Anti-tussitives act on the brain to suppress the cough reflex. Such cough suppressants are used to relieve dry persistent coughs. The most commonly used drugs are dextromethorphan (an NMDA receptor antagonist), codeine and pholcodine (which are opioids. However, one skilled in the art would understand that there are many other well known and common anti-tussitives that may be used. The present invention is optionally direct to the use of anti-tussitves. The anti-tussitive may be used in amounts of less than or equal to 75% of the approved dosage.

### Pharmaceutical Compositions

Compositions of the invention are formulated in a single dosage form, and these may be solid (such as tablets, capsules, sachets, trochets and the like), liquid (such as solutions or suspensions) or inhalation aerosols or patches. While the solid compounds will typically be administered orally, the liquids may be administered orally or by injection. Other dosage forms, such as suppositories, are also useful.

Exemplary compositions of the present invention are directed to solid dosage forms such as bulk powders, tablets, caplets, pellets, capsules, sachets, granules, and any other dosage form suitable for oral administration. For purposes of this specification and the accompanying claims, the term “tablet” refers equally to a tablet, a caplet or any other solid dosage form which is suitable for oral administration.

Binders are agents used to impart cohesive qualities to the powdered material. Binders impart cohesiveness to the tablet formulation which insures the tablet remaining intact after compression, as well as improving the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants have a number of functions in tablet manufacture. They prevent adhesion of the tablet material to the surface of the dies and punches, reduce interparticle friction, facilitate the ejection of the tablets from the die cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. Generally. the lubricant is present in an amount from about 0.25% to about 5% of the weight of the final composition and more specifically from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, aligns, gums and cross-linked polymers. Examples of suitable disintegrants include, but are not limited to, crosscarmelose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. Generally, the disintegrant is present in an amount from about 0.5% to about 25% of the weight of the final composition and more specifically from about 1% to about 15% of the weight of the final composition.

Glidants are substances which improve the flow characteristics of a powder mixture. Examples of glidants include, but are not limited to colloidal silicon dioxide, talc or mixtures thereof. Generally, the glident is present in an amount of from about 0.1% to about 10% of the weight of the final composition and more specifically from 5 about 0.1% to about 5% of the weight of the final composition.

The adsorbent may be, for example colloidal silicon dioxide, microcrystalline cellulose, calcium silicate or mixtures thereof. Generally, the adsorbent is present in an amount from about 0.05% to about 42% of the weight of the final composition and more specifically from about 0.05% to about 37% of the weight of the final composition.

If desired, other ingredients, such as diluents, stabilizers and anti-adherents, conventionally used for pharmaceutical formulations may be included in the present formulations. Optional ingredients include coloring and flavoring agents which are well known in the art.

The pharmaceutical composition described in the present invention may be formulated to release the active ingredients in a sustained release manner. Various formulations, including elixers, suspensions, tablets; caplets, capsules, and the like are contemplated for dosage forms of these components.

The invention is further described by means of the following examples, which are not intended to limit the scope of the claimed invention in any manner.

### EXAMPLE 1

Pharmaceutical compositions dosage forms of the present invention are made of the active ingredients listed below in the following dosage amounts.

TABLE 1

| NSAID | |
|---|---|
| | Decongestant |
| Ibuprofen 200 mg | Pseudoephedrine 30 mg |
| Ibuprofen 400 mg | Pseudoephedrine 60 mg |
| Ibuprofen 200 mg | Pseudoephedrine 30 mg |
| Ibuprofen 200 mg | Pseudoephedrine 30 mg |
| Ibuprofen 200 mg | Phenylpropanolamine HCl 37.5 mg |
| | Antihistamine |
| Ibuprofen 200 mg | Chlorpheniramine 2 mg |
| Ibuprofen 400 mg | Chlorpheniramine 4 mg |
| Ibuprofen 200 mg | Brompheniramine 2 mg |
| Ibuprofen 200 mg | Diphenhydramine HCl 12.5 mg |
| Ibuprofen 200 mg | Clemastine fumarate 0.67 mg |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, articles, publications, and test methods mentioned above are hereby incorporated by reference.

1. A pharmaceutical composition comprising a non-steroidal anti-inflammatory agent and a decongestant and which does not comprise an antihistamine, wherein the amount of decongestant in the composition is less than 75% of an approved dose of decongestant and wherein the NSAID potentiates the activity of the decongestant.

2. The pharmaceutical composition of claim 2, wherein the amount of decongestant is less than 50% of the amount present in an approved dose

3. The pharmaceutical composition of claim 1, wherein the approved dose of decongestant is an adult dose

4. The pharmaceutical composition of claim 1, wherein the approved dose of decongestant is a pediatric approved dose.

5. The pharmaceutical composition of claim 1, wherein the NSAID is ibuprofen and the decongestant is pseudophedrine.

6. The pharmaceutical composition of claim 1, wherein the NSAID is present in the amount of 150 mg to 500 mg and the decongestant is present in the amount of 25 to 75 mg.

7. The pharmaceutical composition of claim 1, wherein the NSAID is ibuprophen present in the amount of about 150 to 250 mg and the decongestant is pseudophedrine present in the amount of about 25 to 35 mg.

8. The pharmaceutical composition of claim 1, wherein the NSAID is ibuprophen present in the amount of about 350 to 450 mg, and the decongestant is pseudophedrine present in the amount of about 55 to 65 mg.

9. A pharmaceutical composition comprising non-steroidal anti-inflammatory agent and an antihistamine and which does not comprise a decongestant, wherein the amount of antihistamine is present in an amount of less than 75% of an approved dose of anti-histamine, and wherein the NSAID potentiates the activity of the anti-histamine.

10. The pharmaceutical composition of claim 9, wherein the amount of antihistamine is present in an amount of less than 50% of an approved dose of anti-histamine.

11. The pharmaceutical composition of claim 9 wherein, the approved dose of antihistamine is an adult dose.

12. The pharmaceutical composition of a claim 9, wherein the approved dose of antihistamine is a pediatric approved dose.

13. The pharmaceutical composition of claim 9, wherein the non-steroidal anti-inflammatory agent is ibuprophen, and the antihistamine is chlorpheniramine.

14. The pharmaceutical composition of claim 9, wherein the non-steroidal anti-inflammatory agent is ibuprophen in the amount of about 150 to 250 mg and the antihistamine is chlorpheniramine present in the amount of about 1 to 3 mg.

15. The pharmaceutical composition of claim 9, wherein the non-steroidal anti-inflammatory agent is ibuprophen in the amount of 350 to 450 mg, and the antihistamine is chlorpheniraine present in the amount of about 1 to 3 mg.

16. A method of relieving symptoms of rhinitis in a mammal, which method comprises administering:
a. an antihistaminic effective amount of an antihistamine, and
b. an anti-inflammatory effective amount of a non-steroidal anti-inflammatory agent, wherein the effective amount of the antihistamine is less than 75% of an amount present in an approved dose of the antihistamine, relative to an amount of the non-steroidal anti-inflammatory agent corresponding to about 100% of the amount present in a normal strength approved dose of the non-steroidal anti-inflammatory agent in the same dosage form, and wherein the antihistamine and the non-steroidal anti-inflammatory agent are present in a single dosage form, and wherein the method does not contemplate administration of an antihistamine.

17. A method of relieving symptoms of rhinitis in a mammal, which method comprises administering:
a. a decongestive effective amount of a decongestant, and
b. an anti-inflammatory effective amount of a non-steroidal anti-inflammatory agent, wherein the effective amount of the decongestant is less than 75% of an amount present in an approved dose of the decongestant, relative to an amount of the non-steroidal anti-inflammatory agent corresponding to about 100% of the amount present in a normal strength approved dose of the non-steroidal anti-inflammatory agent in the same dosage form, and wherein the decongestant and the non-steroidal anti-inflammatory agent are present in a single dosage form, and wherein the method does not contemplate administration of an antihistamine.

18. A method of preparing a pharmaceutical composition, which method comprises admixing therapeutically effective amount of each of:
a. a non-steroidal anti-inflammatory drug and
b. a decongestant, wherein the effective amount of decongestant is less than about 75% an amount present in an approved dose of the decongestant relative to an amount of the non-steroidal anti-inflammatory agent corresponding to about 100% of the amount present in a normal strength approved dosage form of the non-steroidal anti-inflammatory agent, and wherein the composition does not include an antihistamine.

19. A method of preparing a pharmaceutical composition, which method comprises admixing therapeutically effective amounts of each of:
a. a non-steroidal anti-inflammatory agent and
b. an antihistamine, wherein the effective amount of antihistamine is less than about 75% of an amount of amount present in an approved dose of the decongestant relative to an amount of the non-steroidal anti-inflammatory agent corresponding to about 100% of the amount present in a normal strength approved dosage form of the non-steroidal anti-inflammatory agent, and wherein the composition does not include a decongestant.
